(19) United States
(12) Patent Application Publication
(10) Pub. No.: US 2023/0032937 A1
(43) Pub. Date: Feb. 2, 2023

(54) ELASTIC NONWOVEN LAMINATE WITH INELASTIC PARALLEL STRIPS

(71) Applicants: Georg BALDAUF, Bad Bentheim-Gildehaus (DE); Christina OBERHAUS, Berlin (DE)

(72) Inventors: Georg BALDAUF, Bad Bentheim-Gildehaus (DE); Christina OBERHAUS, Berlin (DE)

(21) Appl. No.: 17/877,581

(22) Filed: Jul. 29, 2022

(30) Foreign Application Priority Data
Jul. 29, 2021 (DE) 102021119678.7

(51) Int. Cl. A61F 13/15; A61F 13/56

(52) U.S. Cl. CPC A61F 13/15593; A61F 13/5638; A61F 2013/49022

(57) ABSTRACT

The invention relates to a nonwoven laminated web (20) for making an elastic closure element (57) of a diaper (56) or an elastic shaping element (59) at the edge (58) of a diaper (56), having at least one laminate layer (21 and 22) of nonwoven textile (21 and 22) and a laminate layer (23) of an elastic film (23) adjacent thereto, the laminate layers (21 and 22, 23) being welded to one another at numerous spots (33) in a stretched state of the film (23). During welding, the film (23) is stretched in a central region (43) transverse to the longitudinal direction (L) of the nonwoven laminate (20) and is not stretched at the two longitudinal edge strips (50) in a width of 5-25% of the total width (G) of the film (23) in each case, so that, in the relaxed state of the film (23), the nonwoven laminate (20) forms folds (44) in the nonwoven layer (21 and 22) in the central region (43), which extend in the longitudinal direction (L) of the nonwoven laminate (20), and at the longitudinal edges (50, 51) has no folds in the nonwoven layer (21 and 22) but forms there two parallel, inelastic stiffening zones, the film (23) having a smooth, closed surface structure (49) at the longitudinal edge strips (50) and a wrinkled, furrowed surface structure (47) in the central region (43).

The invention relates to a nonwoven laminated web for making an elastic closure element of a diaper or an elastic shaping element at the edge of a diaper, having at least one laminate layer of nonwoven textile and another laminate layer of an elastic film adjacent thereto, the laminate layers being welded to one another at numerous spots in a stretched state of the film. The invention further relates to the use of a nonwoven laminate according to the invention for making a diaper closure element.

It is known to use a nonwoven laminated web in diapers, where an elastic film is sandwiched between two nonwoven layers and the three laminate layers thus formed are welded together via spot welds. When using such a nonwoven laminate in diapers, it is important that the hygiene product is easy to manufacture and that the lateral edges of the laminate can be easily and securely fastened to one another.

It is the object of the invention to achieve a high level of reliability in the processing and fastening of the laminate to form elastic diaper closure elements or elastic shaping elements at the edge of a diaper. It is also the object of the invention to give an impression of softness and drapability in the final product.

These objects are attained according to the invention in that the film is stretched during welding in a central region transverse to the longitudinal direction of the nonwoven laminate and is not stretched at the two longitudinal edges in a width of in each case 5-25% of the total width of the film, so that in the relaxed state of the film the nonwoven laminate forms folds in the nonwoven layer in the central region that extend in the longitudinal direction of the nonwoven laminate, and at the longitudinal edges has no folds in the nonwoven layer but forms there two parallel, inelastic stiffening zones, the film having a smooth, closed surface structure at the longitudinal edges and a wrinkled, furrowed surface structure in the central region.

Advantageous embodiments of the nonwoven laminate according to the invention are listed in the subclaims.

The advantage of the design of an elastic laminate with two parallel, inelastic longitudinal edges adjacent to the elastic area is the higher process reliability when processing the laminate into elastic diaper closure elements and their simpler and more secure attachment to the diaper body, as will become clear below.

The processing of the nonwoven laminate according to the invention into diaper closure elements is carried out by the steps of:

Conveying the nonwoven laminate longitudinally to a stationary first station where adhesive closures are repeatedly applied to one of the inelastic longitudinal edges of the nonwoven laminate in a flag-like manner projecting therefrom so that there is a series of spaced adhesive closures,

Conveying the nonwoven laminate longitudinally to a stationary second station where the desired closure elements are repeatedly punched out of the nonwoven laminate such that each closure element comprises one of the adhesive closures; and

Fastening the closure elements with their inelastic longitudinal edge strip opposite the adhesive closure to a diaper body, in particular by adhesive and/or a pressing and/or ultrasonic welding.

In both fastening processes, i.e. for the attachment of the adhesive fasteners to the nonwoven laminate on the one hand and the attachment of the fastening element punched out from the latter to a diaper body, a firm laminate composition in the fastening zone that is not softly elastically deformable is advantageous. For this reason, it is provided in accordance with the invention that the inelastic longitudinal edges of the nonwoven laminate form the fastening zones.

Another advantage of the nonwoven laminate according to the invention is that the increased stiffness of the longitudinal edges compared to the elastic central region results in improved processability on a diaper machine on which the above steps are carried out, since the laminate has a lower curl, i.e. less tendency to curl toward the center of the web.

In one embodiment, the nonwoven laminate has only one nonwoven laminate layer. Preferably, however, it has two nonwoven textile layers, with the film between them. Thus, both sides of the laminate are made of nonwoven textile and are thus soft and conformable, which is particularly desirable for hygiene products.

The film can be a coextruded film with a layer structure of type A/B/A, where the thickness of layer A can be between 2% and 14% of the total thickness of the film. This improves the tear resistance.

The film can occupy 10% to 100% of the total area in the nonwoven laminate, preferably 25% to 50%.

The spots are preferably in sinusoidal rows, the amplitudes of which may be for of example 0.2 to 0.5 times the spacing between the rows in the longitudinal direction of the nonwoven laminate. By forming sinusoidal rows, more spots can be along a row at the same spacing from each other than in a straight row, so that more attachment points hold the laminate layers together. Another advantage of using sinusoidal rows is that the ultrasonic device used to create the weld spots wears less. To this end, it may be shown that the welding spots can be generated by welding bumps on a welding roller against which the laminate to be welded rests, with a sonotrode opposite the welding roller to apply ultrasonic vibrations to the welding bumps to heat them, with the laminate lying between the sonotrode and the welding roller. A sinusoidal arrangement of the welding bumps along the roller axis ensures that at least one welding bump is always opposite the sonotrode, and not alternately none and all of the welding bumps, as is the case when a welding roller with straight rows of welding bumps rotates.

The sinusoidal arrangement of the weld studs creates a clearly visible, wave-like structure in the nonwoven laminate after the film has been relaxed, giving the appearance that a product made from it has high softness and optimum drapeability. In this regard, the process is characterized by simple manufacturing steps and by easy further processing into the final product.

The spacing between the rows of weld spots in the longitudinal direction of the nonwoven laminate can be between 2 mm and 20 mm preferably between 3 mm to 5 mm.

The weld spots along the rows can be created with a spacing from each other of 2 mm to 4 mm, preferably of 2.2 to 2.6 mm.

The laminate layer or layers of nonwoven textile may or may not each have a weight of 10 g/m² to 30 g/m².

The film can have a weight from 25 g/m² to 110 g/m².

To make the web-like nonwoven laminate, the film can be stretched in the central region transverse to the longitudinal direction of the nonwoven laminate by 1.5 to 3 times, in particular by 2.6 to 2.8 times, and not stretched at the two longitudinal edges in a width of 5-25% of the total width of the film in each case, the laminate layers then being welded in this state.

The laminate layers can be held on the welding roll during welding by a vacuum. For this purpose, the welding roll can have suction openings through which the nonwoven laminate is sucked in.

Examples of embodiments of the invention are described in more detail below. In the drawing:

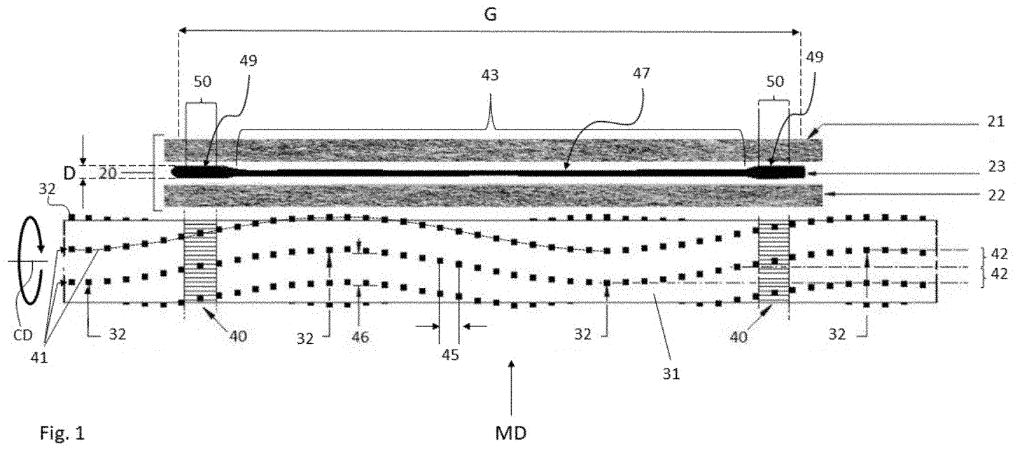

FIG. 1 is a schematic cross-section through a nonwoven laminated web according to the invention transverse to the direction of web travel with a welding roller under the laminate;

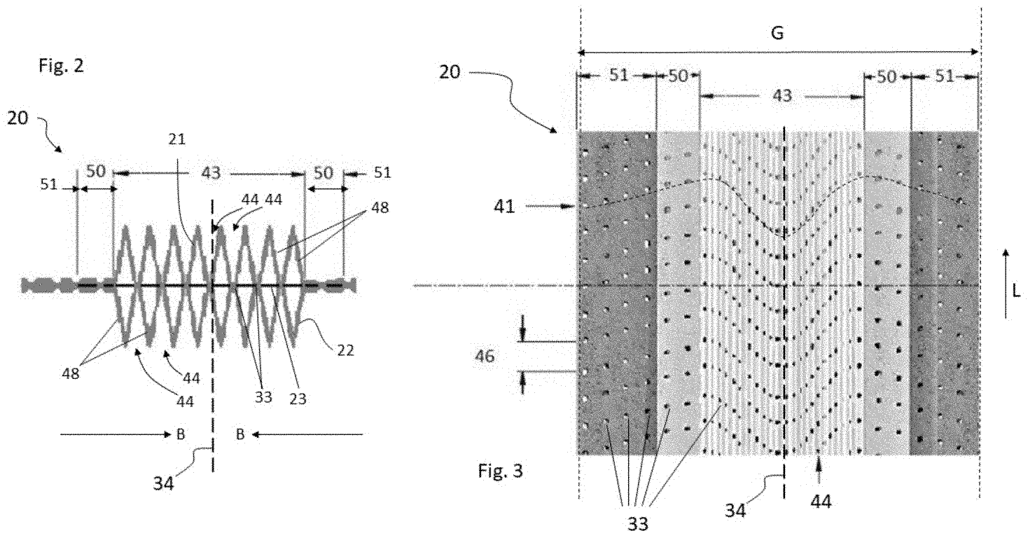

FIG. 2 is a cross-section through the nonwoven laminate after the elastic film has been relaxed, folds of the nonwovens being shown schematically;

FIG. 3 is a top view of the nonwoven laminate after when relaxed;

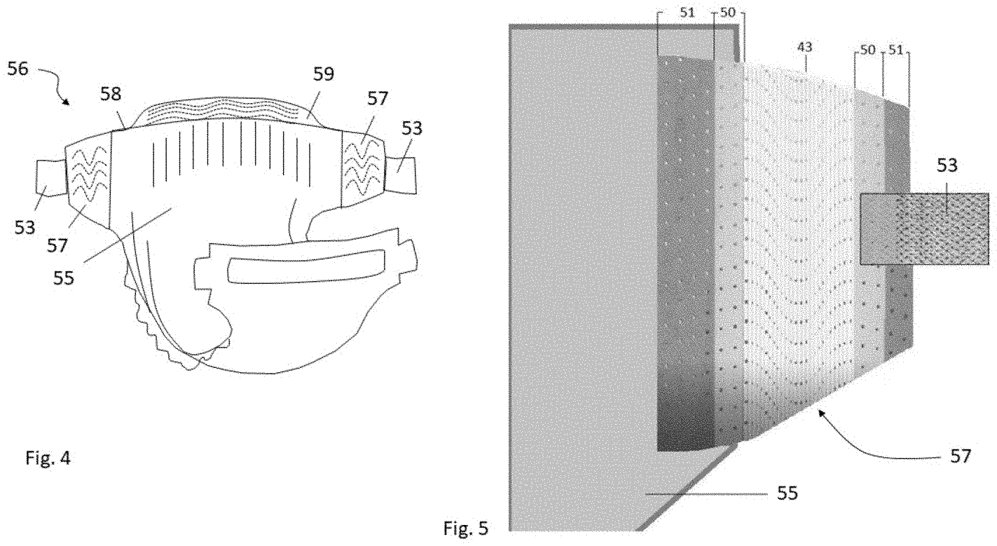

FIG. 4 is a schematic view of a diaper;

FIG. 5 is a top view of a diaper closure element made from the nonwoven laminate on a diaper body

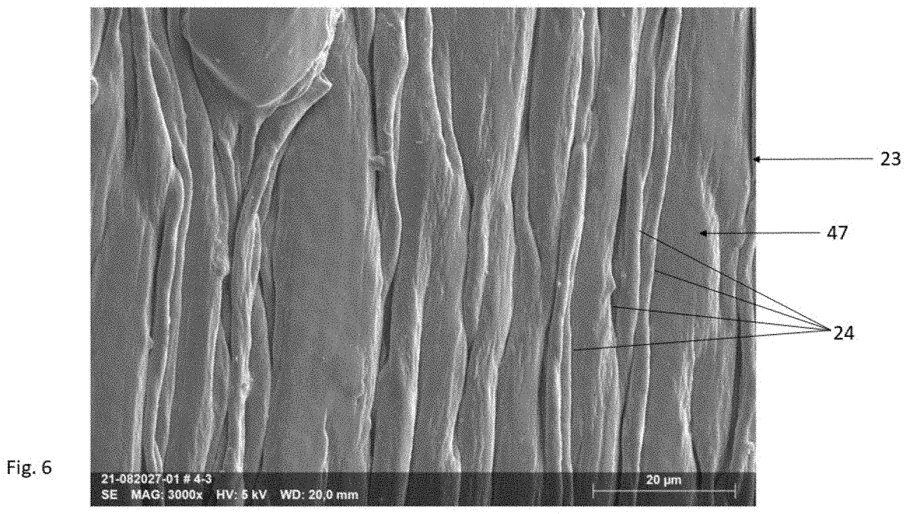

FIG. 6 is a top view of the film surface in the elastic region in the relaxed state after stretching;

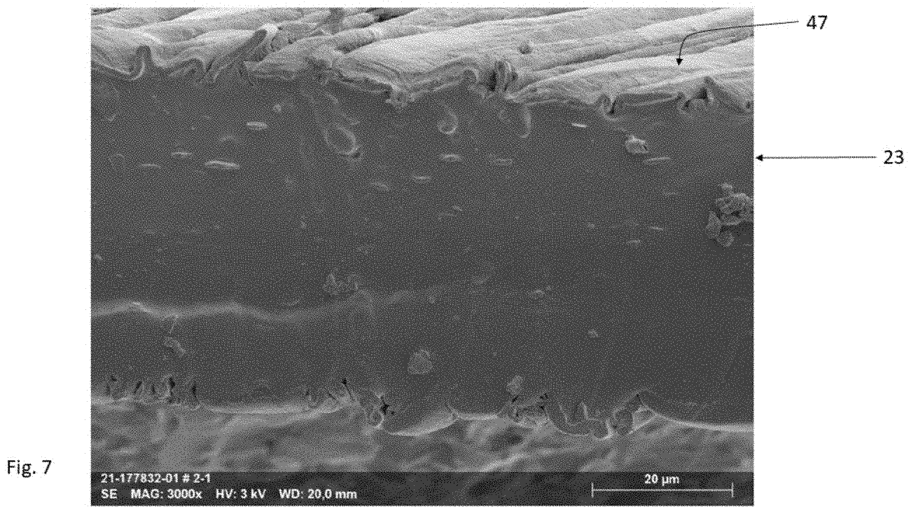

FIG. 7 is a cross-section through the film in the elastic region transverse to the longitudinal direction in the relaxed state after stretching;

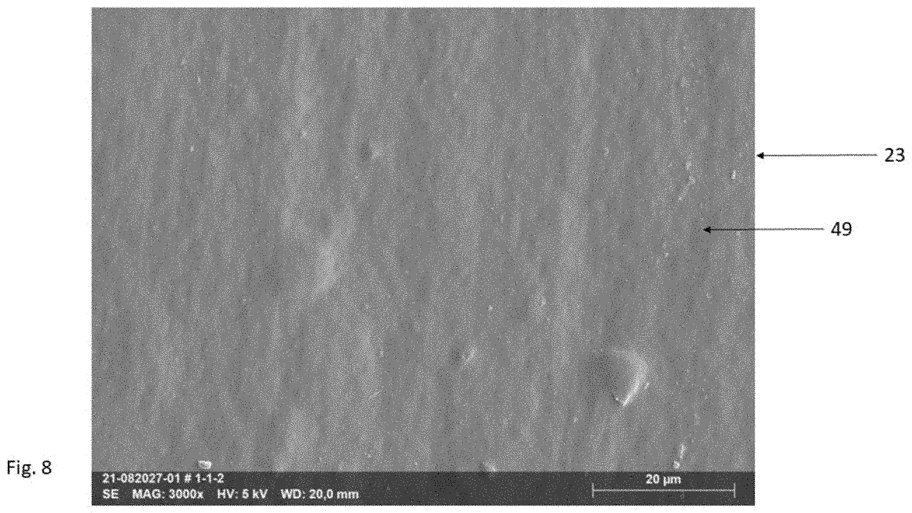

FIG. 8 is a top view of the film surface in the area of the side edge;

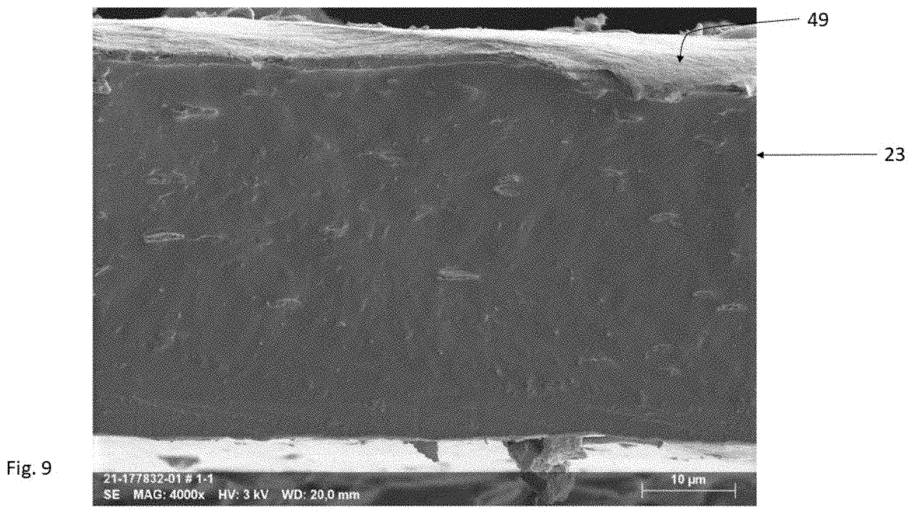

FIG. 9 is a cross-section through the film surface in the area of the side edge transverse to the longitudinal direction; and

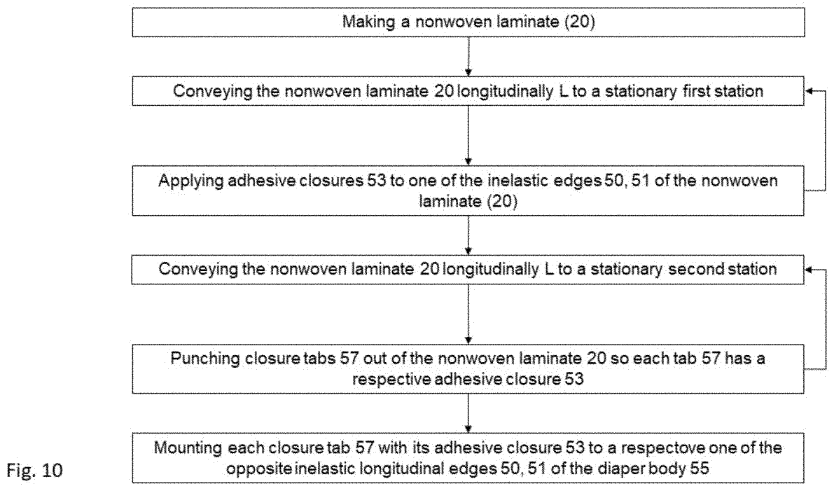

FIG. 10 is a process diagram for making a diaper closure element,

FIG. 1 shows a nonwoven laminated web 20 that in this embodiment has two laminate layers 21 and 22 of a nonwoven textile and a laminate layer 23 of an elastic film. An alternative nonwoven textile laminate differs from this nonwoven textile laminate 20 in that only one nonwoven textile layer is used rather than two so that only one face of the elastic film 23 carries a nonwoven layer 21. However, the properties of this alternative nonwoven laminate are identical to the nonwoven laminate 20 described below.

In a stretched state of the film 23, the laminate layers 21, 22, and 23 are welded to one another at numerous spots 33 while the film 23 is stretched only in a central region 43 transverse to the longitudinal direction L of the nonwoven laminate 20 and not stretched at two longitudinal edge strips 50 in a width of in each case 5-25% of the total width G of the film 23. The nonwoven laminate 20 thus forms folds 44 in the nonwoven 21 and 22 in the central region 43 in the relaxed state of the film 23 when it contracts in the direction B toward the web center 34 as shown in FIG. 2. This is because the spot welding forms thick regions 48 in the two nonwoven layers 21 and 22 between two adjacent welding points 33, and the above-described folds 44 are formed between the thick regions 48. The thick regions 48 form a material reservoir of the nonwoven layers 21 and 22 that makes the laminate 20 stretchable overall despite the inelastic nonwoven layers in the central region 43. The folds 44 extend in the longitudinal direction L of the nonwoven laminate 20 as shown in FIG. 3.

At the longitudinal edges 50, 51, on the other hand, the laminate 20 does not have any folds 44 in the nonwovens 21 and 22. Rather, there are inelastic stiffening zones running longitudinally parallel to the web center 34. At the longitudinal edge strips 50, i.e. at both the right and left edges 50, the film 23 has a smooth, closed surface structure 49 shown in FIG. 8, while in the central region 43 there is a wrinkled, furrowed surface structure 47 shown in FIG. 6. FIG. 7 shows a cross-section through the film 23 in the elastic region 43 transverse to the longitudinal direction L. It also reveals the wrinkled, furrowed surface structure 47. Correspondingly, FIG. 9 shows a cross-section through the foil 23 at a side edge 50 transverse to the longitudinal direction L. It reveals the smooth, closed surface structure 49. As a result of this smooth, closed surface structure 49 and the fact that the longitudinal edge strips 50 have not been stretched during welding, the longitudinal edge strips 50 are particularly suitable as fastening zones.

In the embodiment according to FIG. 1, the film 23 extends only over part of the total width G of the nonwoven laminate 20, so that the nonwoven layers 21 and 22 project laterally beyond the film 23 and form outer longitudinal edge strips 51 in this projecting region where they lie directly on top of one another. In other words, no film 23 lies at the outer longitudinal edge strips 51 between the nonwovens 21 and 22. In the direction toward the web center 34, each inner longitudinal edge strip 50 lies between the central region 43 and the respective outer longitudinal edge strips 51 and there the film 23 lies between the nonwovens 21 and 22.

To produce the web-like nonwoven laminate 20, the film 23 is stretched between the two nonwoven layers 23 in the central region 43 transversely to the longitudinal direction L of the nonwoven laminate 20 μm by 1.5 to 3 times (50 to 200%), in particular by 2.6 to 2.8 times, and is not stretched at either of the two longitudinal edge strips 50 in a width of 5-25% of the total width G of the film 23. Subsequently, with the central film 23 stretched, the laminate layers 21 and 22, 23 are placed on a welding roller 31 and welded in this state.

The welding roller 31 is shown in FIG. 1 below the nonwoven laminate 20. It rotates in the web-travel direction MD. The longitudinal direction L of the nonwoven laminate 20 corresponds to its web-travel direction MD. The axial direction is shown at CD in FIG. 1.

The welding roller 31 has suction openings 40 through which the nonwoven laminate 20 is pulled in, i.e. held on the roller 31 by a vacuum, while the laminate 20 is welded. This is done by ultrasound.

The nonwovens 21 and 22 consist of polymer fibers, preferably polyolefin. The elastic film 23 consists, for example, of PE (polyethylene), of SIS (felt or sisal), of SBS7, i.e. a painter's fleece or cover fleece with a PE film, of SEBS (styrene-ethylene-butylene-styrene) or of POE (polyolefin elastomer).

The roller 31 has raised welding bumps 32 that are heated by a sonotrode opposite them on the other side of the nonwoven laminate 20, not shown in FIG. 1, with ultrasonic vibration, depending on which welding nubs or bumps are opposite due to the rotation of the welding roller 31. Due to the ultrasonic heating of the welding bumps 32, the film melts at these locations and the melted material of the film 23 penetrates into the nonwoven laminates 21 and 22 and spot-welds them after solidification, so that each welding bump 32 of the roller 31 creates a welding spot 33 in the nonwoven laminate where the laminate layers are welded together.

The weld bumps 32 are in rows 41 that are transverse to the web-travel direction MD, in other words in the axial direction CD of the welding roll 31. Furthermore, the weld studs 32 form sinusoidal waves with an amplitude 42 that is 0.2 to 0.5 times the spacing 46 of the weld spot rows 41 from each other in the web-travel direction MD. Here, the spacing 46 of the rows of weld spots 41 from one another in the web-travel direction MD is 2 mm to 20 mm, preferably 3 mm to 5 mm. The weld studs 32 have a spacing 45 from one another on the welding roller 31 within a weld spot row 41 of 2 mm to 4 mm, preferably of 2.2 mm to 2.6 mm.

As soon as the laminate 20 is removed from the welding roller 31, the elastic film 23 contracts and creates the folds 44 in both nonwoven layers 21 and 22, i.e. on both sides of the film 23, the folds 44 being substantially parallel to each other. Because of these folds 44, the laminate is stretchable for use, for example, in hygiene products, in particular as an elastic diaper closure 57 in a baby diaper 56.

In the unstretched, relaxed state, the wave shape is particularly visible due to the shorter wavelength, i.e. the wave-like structure is shown to its best advantage in the product and symbolizes a high degree of softness and drapability.

The nonwoven layers 21 and 22 have a weight of 10 to 30 g/m² and the elastic film 23 has a weight of 30 to 110 g/m². The film 23 occupies 25 to 100 percent of the total area of the laminate.

The film 23 is preferably a coextruded film with a layer structure of the type A/B/A, where the thickness of the layer A at the two side edges 50 is between 2%-14% of the total thickness D of the film 23. The two side edges 50 each have a width of 5-25% of the total width G of the film 23 and are not stretched.

Definition of elastic: The laminate 20 is stretched to 100% of its length transverse to the longitudinal direction and after stretching shows an elastic recovery of at least 80% and thus a plastic deformation of at most 20%, more precisely in the range of 5%-10%. A measuring method of determining the hysteresis is described in international application WO 2018/031841 [U.S. Pat. No. 11,446,186].

As already explained, the laminate according to the invention is suitable for making an elastic closure element 57 of a diaper 56 or an elastic shaping element 59 at the edge 58 of a diaper 56, for example for babies or toddlers, as schematically shown in FIG. 4. FIG. 5 shows the side edge of the diaper 56 in the waist area to which a closure element 57 is attached. This is done by the following steps that can be carried out on a so-called diaper machine and are shown in FIG. 10:
Providing a nonwoven web laminate 20 according to the invention.
Conveying the nonwoven laminate 20 longitudinally L to a stationary first station where adhesive closures 53 are repeatedly applied to one of the inelastic longitudinal edges 50, 51 of the nonwoven laminate 20 in a flag-like manner projecting therefrom so that there is a series of spaced adhesive closures 53,
Conveying the nonwoven laminate 20 longitudinally to a stationary second station where the desired closure elements 57 are repeatedly punched out of the nonwoven laminate 20 such that each closure element 57 comprises one of the adhesive closures 53; and
Fastening the closure elements 57 with their inelastic longitudinal edge strips 50, 51 opposite the adhesive closure 53 to a diaper body 55, in particular by adhesive and/or a pressing and/or ultrasonic welding.

Due to the stiff, non-soft elastically deformable edges 50, 51 that here form the fastening zones, fastening of the adhesive fasteners 53 to the nonwoven laminate 20 on the one hand and the fastening of the diaper fastening element 57 made from the latter to the diaper body 55 on the other hand, is particularly easy to manufacture and process-safe.

### LIST OF REFERENCE SIGNS

20 Nonwoven laminate
21 First laminate layer of nonwoven textile
22 Second laminate layer of nonwoven textile
23 Elastic film
24 Furrows in foil surface
31 Welding roller
32 Welding bumps
33 Welding point
34 Railway center
40 Suction openings
41 Series
42 Welding spot amplitude
43 Middle area of the elastic film
44 Folds in nonwoven
45 Bump spacing along one row
46 Row spacing
47 Surface structure of the film inside, wrinkled, furrowed
48 Thickened region
49 Surface structure of the film outside, smooth, closed
50 Inner longitudinal edge strip
51 Outer longitudinal edge strip
53 Adhesive closure
55 Diaper body
56 Diaper
57 Closure element/Diaper closure
58 Border
59 Elastic shaping element
MD Web-travel direction
CD Axis direction of the welding roll, perpendicular to MD
L longitudinal direction
G Total width of the film
D Total thickness of the film
B Relaxation direction

1. A nonwoven laminated web for making an elastic closure element of a diaper or an elastic shaping element at the edge of a diaper comprising:
at least one laminate layer of nonwoven; and
a laminate layer of an elastic film adjacent thereto, the laminate layers being welded to one another at numerous spots in a stretched state of the film, during welding, the film being stretched in a central region transverse to the longitudinal direction of the nonwoven laminate and not stretched at two longitudinal edge strips in a width region of in each case 5-25% of the total width of the film so that, in the relaxed state of the film, the nonwoven laminate forms in a central region of the nonwoven textile folds that extend in the longitudinal direction of the nonwoven laminate, and has no folds in the nonwoven layer at the longitudinal edges, but forms two parallel, inelastic stiffening zones there, the film having a smooth, closed surface structure at the longitudinal edge strips and a wrinkled, furrowed surface structure in the central region.

2. The nonwoven laminate according to claim 1, wherein there are two of the laminate layers of nonwoven and the film is sandwiched therebetween.

3. The nonwoven laminate according to claim 1, wherein the film is a coextruded film having a layer structure of the type A/B/A, the thickness of layer A being between 2% and 14% of a total thickness of the film.

4. The nonwoven laminate according to claim 1, wherein the film occupies 10% to 90% of the total area in the nonwoven laminate, preferably 25% to 50%.

5. The nonwoven laminate according to claim 1, wherein the welding spots are in sinusoidal rows whose amplitudes are 0.2 to 0.5 times a spacing between the rows in a longitudinal direction of the nonwoven laminate.

6. The nonwoven laminate according to claim 5, wherein the spacing between the rows of weld spots in the longitudinal direction of the nonwoven laminate is between 2 mm and 20 mm, preferably between 3 mm and 5 mm.

7. The nonwoven laminate according to claim 5, wherein the weld spots are produced along the rows with a spacing from each other of from 2 mm to 4 mm.

8. The nonwoven laminate according to claim 1, wherein the laminate layer of nonwoven has a weight of 10 g/m² to 30 g/m².

9. The nonwoven laminate according to claim 1, wherein the film has a weight of 25 g/m² to 110 g/m².

10. A method of making the web-like nonwoven laminate according to claim 1, wherein the film is stretched in a central region transverse to the longitudinal direction f the nonwoven laminate by 1.5 to 3 times and is not stretched at two longitudinal edge strips each of a width of 5-25% of a total width of the film, and the laminate layers are subsequently welded to each other in this state.

11. The method according to claim 10, wherein the laminate layers are held on a welding roller by vacuum during welding.

12. Use of the web-like nonwoven laminate according to claim 1 for making closure elements for diapers, characterized by the steps of:
conveying the nonwoven laminate longitudinally to a stationary first station where adhesive closures are repeatedly applied to one of the inelastic longitudinal edges of the nonwoven laminate in a flag-like manner projecting therefrom so that there is a series of spaced-apart adhesive closures,
conveying the nonwoven laminate longitudinally to a stationary second station where the desired closure elements are repeatedly punched out of the nonwoven laminate such that each closure element comprises one of the adhesive closures; and
fastening the closure elements with their inelastic longitudinal edge strip opposite the adhesive closure to a diaper body, in particular by adhesive and/or a pressing and/or ultrasonic welding.

13. A method of making a diaper comprising the steps of sequentially:
juxtaposing a longitudinally extending textile nonwoven layer of predetermined transverse width with an elastic film to form a longitudinally extending laminate having two longitudinally extending outer edges;
transversely stretching a central region of the elastic film while leaving unstretched edge regions of the elastic film each having a transverse width equal to between 5% and 25% of the predetermined transverse width;
welding the nonwoven layer to the elastic film while the central region is stretched and the edge regions are unstretched at a multiplicity of transversely and longitudinally spaced spots;
relaxing the stretched central region such that the laminate forms in the central region a plurality of longitudinally extending folds while the edge regions remain generally smooth;
attaching to at least one of the outer edges a succession of longitudinally spaced fastener tabs extending transversely from the one outer edge;
punching out from the one outer edge pieces of the laminate each carrying a respective one of the edge; and
fastening each of the punched-out pieces to a respective edge of a diaper body.

14. The method defined in claim 13, wherein the punched-out pieces are fastened to the diaper body by the respective other edges.
